(19) United States
(12) Patent Application Publication
(10) Pub. No.: US 2023/0112472 A1
(43) Pub. Date: Apr. 13, 2023

(54) ELECTROCARDIOGRAM DETECTION MODULE WITH ENLARGED AREA OF CONTACT AND IMPROVED DURABILITY FOR MOUNTING ON STEERING WHEEL, AND STEERING WHEEL

(71) Applicant: Mobile Drive Netherlands B.V., Amsterdam (NL)

(72) Inventor: JYUN-WEI CHEN, New Taipei (TW)

(21) Appl. No.: 17/963,294

(22) Filed: Oct. 11, 2022

(30) Foreign Application Priority Data
Oct. 13, 2021 (CN) 202111194345.1

(51) Int. Cl. A61B 5/28; A61B 5/26; A61B 5/00

(52) U.S. Cl. CPC A61B 5/28; A61B 5/26; A61B 5/6893

(57) ABSTRACT

An electrocardiogram (ECG) detection module with longer service life includes an ECG detection device, the ECG detection device includes a conductive adhesive film, and a support member. Bioelectric signals of both hands of a driver/user are obtained by the conductive adhesive film arranged on a surface of the steering wheel. The support member is elastic and arranged between the steering wheel and the conductive adhesive film. The support member causes the conductive adhesive film to adhere to the contours of the steering wheel, and the support member further increases and enlarges area of contact between the conductive adhesive film and the hand of the user. The present disclosure also provides a steering wheel incorporating the detection module.

### TECHNICAL FIELD

The present disclosure relates to the technical field of auto parts, in particular to electrocardiogram (ECG) detection module and steering wheel.

### BACKGROUND

An automobile steering wheel is a good location for mounting an electrocardiogram (ECG) detection unit to detect ECG of the driver. The state of health of the heart of the driver can be detected through the ECG information. When the driver has symptoms such as arrhythmia or atrial fibrillation, the driver can be reminded to take measures in time to avoid accidents. The steering wheel ECG detection technology often uses electrodes to collect bioelectric signals. However, the electrodes themselves are easily oxidized and the conductive layer can easily fall off.

Therefore, improvement is desired.

### SUMMARY OF THE DISCLOSURE

The present disclosure aims to provide an electrocardiogram (ECG) detection module and steering wheel, the conductive adhesive film can be used for bioelectric signal acquisition, the present disclosure adopts an integrated design and can increase the service life of the ECG detection module.

The embodiment of the present disclosure provides an electrocardiogram (ECG) detection module, the ECG detection module includes an ECG detection device, the ECG detection device includes:
a conductive adhesive film arranged on a surface of the steering wheel and configured to obtain a bioelectric signal of a hand of a user; and
a support member arranged between the steering wheel and the conductive adhesive film, configured to cause the conductive adhesive film to adhere to the steering wheel, and configured to increase a contact area between the conductive adhesive film and the hand of the user. The support member is elastic.

In some embodiments, the support member comprises a substrate, the substrate is arranged between the conductive adhesive film and the steering wheel, and the base material is configured to cause the conductive adhesive film to adhere to the steering wheel.

In some embodiments, the support member further comprises an elastic material, the elastic material is arranged between the substrate and the steering wheel and configured to increase an area of contact between the conductive adhesive film and the hand of the user.

In some embodiments, the conductive adhesive film is imprinted on the support member by heat transfer.

In some embodiments, the ECG detection device is stretched by thermoforming and laid on the steering wheel.

In some embodiments, the ECG detection device further comprises an impedance measuring circuit, the impedance measuring circuit is connected to the conductive adhesive film, and the impedance measuring circuit is configured to obtain a biological impedance signal according to the bioelectric signal.

In some embodiments, the ECG detection device further comprises an amplifier, the amplifier is connected to the impedance measurement circuit, and the amplifier is configured to amplify the biological impedance signal.

In some embodiments, the ECG detection device further comprises a processor, and the processor is connected to the amplifier and configured to obtain a biological ECG according to the biological impedance signal.

The embodiment of the present disclosure further provides a steering wheel, the steering wheel includes a main body and an ECG detection module as described above.

In some embodiments, the ECG detection device is symmetrically arranged in more than one part of the steering wheel, remaining areas of the steering wheel are provided with a coating material.

In the present disclosure, the conductive adhesive film can be used for bioelectric signal acquisition, the present disclosure adopts an integrated design and can increase the service life of the ECG detection module.

### BRIEF DESCRIPTION OF THE DRAWINGS

FIG. 1 is a schematic diagram of a steering wheel according to an embodiment of the present disclosure.

FIG. 2 is a schematic diagram of an ECG detection device according to an embodiment of the present disclosure.

FIG. 3 is a schematic diagram of an ECG detection module and an on-board device according to an embodiment of the present disclosure.

### DETAILED DESCRIPTION

The technical solutions in the embodiments of the present disclosure will be described in conjunction with the accompanying drawings in the embodiments of the present disclosure. Obviously, the described embodiments are part of the embodiments of the present disclosure, not all of them. Based on the embodiments of the present disclosure, all other embodiments obtained by those of ordinary skill in the art without creative work shall fall within the protection scope of the present disclosure. The terms used in the description of the present disclosure herein are only for the purpose of describing specific embodiments and are not intended to limit the present disclosure.

In the embodiment of the present disclosure, “at least one” refers to one or more, and “multiple” refers to two or more. Unless otherwise defined, all technical and scientific terms used herein have the same meanings as those commonly understood by those skilled in the technical field in the present disclosure. The terms used in the specification of the present disclosure are only for the purpose of describing specific embodiments and are not intended to limit the present disclosure.

In the embodiment of the present disclosure, “first”, “second” and other numerical modifiers are only used for the purpose of distinguishing descriptions and cannot be understood as indicating or implying relative importance, or as indicating or implying order. The features defined as “first” and “second” may include one or more of the features explicitly or implicitly. In the description of the embodiments of the present disclosure, the words “exemplary” or “for example” are used as examples, examples or explanations. Any embodiment or design described as “exemplary” or “for example” in the embodiments of the present disclosure shall not be interpreted as being more preferred or advantageous than other embodiments or designs.

The steering wheel ECG detection technology in related technologies often uses electrodes to collect bioelectric signals related to the health of a driver’s heart and indicators of other health-states of the driver. However, the electrodes themselves are easy to be oxidized and the conductive layer can easily fall off.

For example, ECG steering wheel often adopts electrode ECG acquisition or optical ECG acquisition. The electrode ECG acquisition uses metal electrode, metal conductive cloth, or metal copper foil to collect the bioelectric signals. The optical ECG acquisition uses infrared light or green light to collect bioelectric signals. The application of the metal electrode to collect the bioelectric signals is challenged by hard texture, poor comfort, and easy oxidation of metal surface, which affects the measurement results. The application of the metal conductive cloth to collect the bioelectric signals suffers from the tin plating on the surface of the conductive cloth falling off, and the hands of the user will be contaminated with smell of metal, affecting the user experience. The application of the metal copper foil to collect the bioelectric signals has problems of soft texture, easy to wrinkle, and easy oxidation and discoloration. The application of the optical ECG to collect ECG signals has the problem that the measurable area is small and the user needs to adjust his mode of holding the steering wheel, and the light issued by the optical ECG may affect the driving safety of the user.

The ECG detection module and steering wheel provided by the embodiment of the present disclosure are described below in combination with the accompanying drawings.

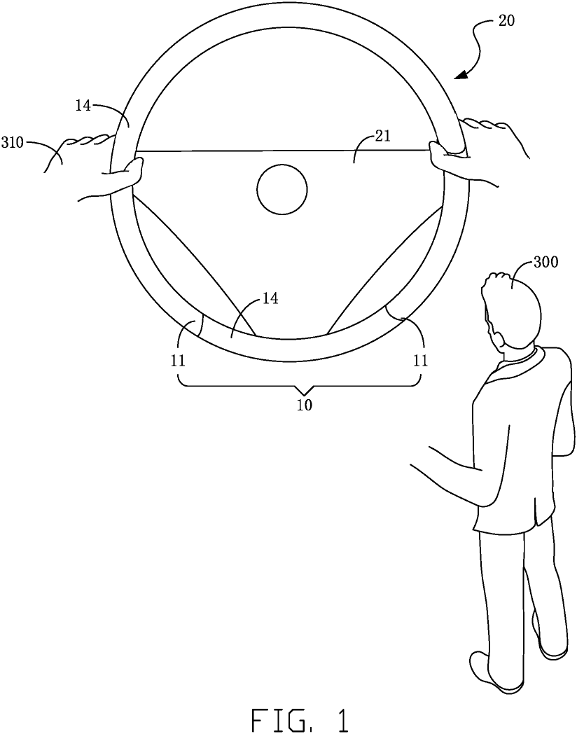

FIG. 1 illustrates a steering wheel 20 in accordance with an embodiment of the present disclosure.

The steering wheel 20 includes a main body 21 and an ECG detection module 10.

In the embodiment, the ECG detection module 10 includes two ECG detection devices 11. The ECG technology needs to collect bioelectric signals from different positions on the body surface of the user, and then amplify the bioelectric signals and record ECG patterns through the signal processing module. Therefore, the ECG detection module 10 needs to include at least two ECG detection devices 11 to obtain bioelectric signals from the hands 310 (such as left hand and the right hand) of the user 300. There is a certain interval between the two ECG detection devices 11 to avoid the mutual interference of bioelectrical signals from the hands 310 of the user 300.

The ECG detection device 11 can be symmetrically arranged in areas of the steering wheel 20, corresponding to the placement positions of the left hand and the right hand of the user on the steering wheel when driving the vehicle, so as to obtain the bioelectric signals from the left hand and the right hand of the user. The areas where the ECG detection device 11 is not provided on the steering wheel 20 may have a coating material.

The steering wheel 20 can use the ECG detection device 11 for ECG signal acquisition. The ECG detection device 11 adopts an integrated design, which can increase the service life of the ECG detection module 10 and improve the user experience.

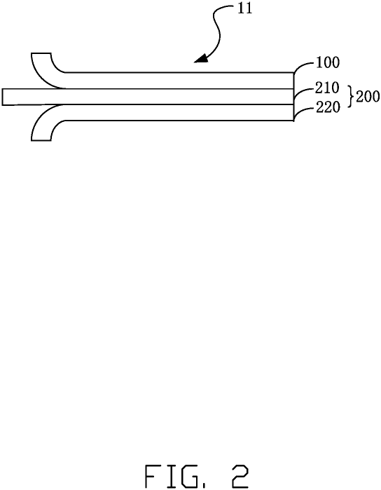

FIG. 2 illustrates an ECG detection device 11 in accordance with an embodiment of the present disclosure.

The ECG detection device 11 includes a conductive adhesive film 100 and a support member 200. The conductive adhesive film 100 is arranged on the surface of the steering wheel 20. The conductive adhesive film 100 obtains and transmits the bioelectric signal of the hand of the user. In one embodiment, the conductive adhesive film 100 may be a carbon conductive adhesive film.

The support member 200 is arranged between the conductive adhesive film 100 and the steering wheel 20. The support member 200 is elastic so that the conductive adhesive film 100 can adhere to the steering wheel 20. The support member 200 is also used to increase the contact area between the conductive adhesive film 100 and the hand of the user.

The support member 200 includes a substrate 210 and an elastic material 220. The substrate 210 is arranged adjacent to the conductive adhesive film 100, and the elastic material 220 is arranged adjacent to the side of the steering wheel 20. The substrate 210 is a material with high extensibility. The substrate 210 is used to provide support for the conductive adhesive film 100, so that the conductive adhesive film 100 can adhere to the steering wheel 20. For example, the substrate 210 may be a cloth substrate with high extensibility.

The elastic material 220 is a material with good elasticity. The elastic material 220 provides a good hand grip for the ECG detection device 11 and can increase the contact area between the conductive adhesive film 100 and the hand of the user. For example, the elastic material 220 may be an elastic sponge material.

In the embodiment, the carbon conductive adhesive film can be imprinted on a transfer substrate by thermal ironing transfer technology to form the conductive adhesive film 100. For example, the extensibility of the conductive adhesive film 100 on the transfer substrate may be improved by thermal ironing transfer.

The conductive adhesive film 100, the substrate 210, and the elastic material 220 can be made into an integrated ECG detection device 11 by a thermoforming process, and the ECG detection device 11 is laid on the steering wheel 20 by stretching, cutting, and sewing.

The ECG detection device 11 may be symmetrically arranged on partial areas of the steering wheel 20. The remaining areas where the ECG detection device 11 is not provided on the steering wheel 20 may have a coating material 14.

The ECG detection device 11 can be integrally formed as one body, so as to adhere to the hand of the user. The ECG detection device 11 can detect the bioelectrical signal of the user, and the ECG detection module 10 can analyze the bioelectrical signal obtained by the ECG detection device 11 to obtain the ECG signal.

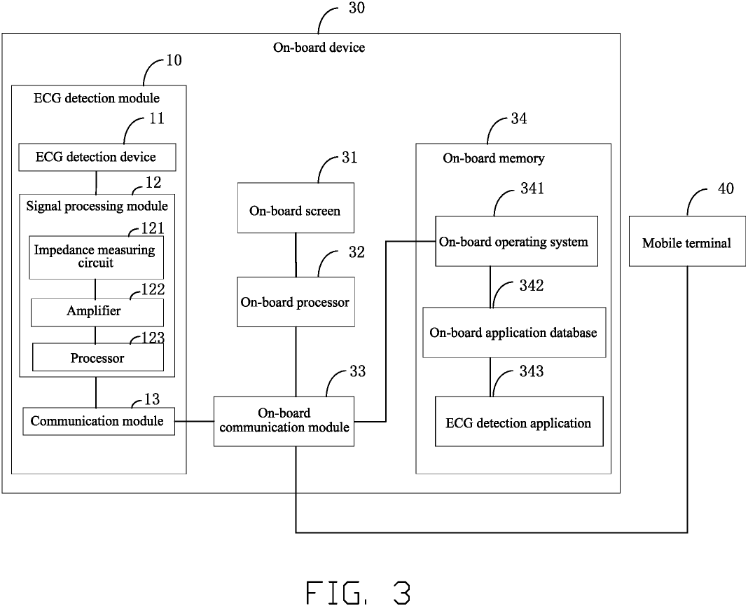

FIG. 3 is a schematic diagram of the ECG detection module 10 applied to an on-board device 30 according to an embodiment of the present disclosure.

The ECG detection module 10 further includes a signal processing module 12 and a communication module 13. The ECG detection device 11 is connected to the signal processing module 12 and the communication module 13. The ECG detection module 10 is disposed in the on-board device 30.

The on-board device 30 further includes an on-board screen 31, an on-board processor 32, an on-board communication module 33, and an on-board memory 34. The on-board screen 31 is connected to the on-board processor 32 and the on-board communication module 33.

The on-board memory 34 includes an on-board operating system 341, an on-board application database 342, and an ECG detection application 343. The on-board communication module 33 is connected to the on-board operating system 341. The on-board operating system 341 is connected to the on-board application database 342 and the ECG detection application 343. The on-board device 30 is further connected to the mobile terminal 40 through the on-board communication module 33.

The signal processing module 12 is used to receive the bioelectric signal from the ECG detection device 11, and amplify and analyze the bioelectric signal to obtain the ECG signal. The communication module 13 is connected to the on-board communication module 33 to transmit the ECG signal to the on-board communication module 33.

The signal processing module 12 includes an impedance measuring circuit 121, an amplifier 122, and a processor 123.

The impedance measuring circuit 121 is connected to the conductive adhesive film 100. The impedance measuring circuit 121 is used to obtain a biological impedance signal according to the bioelectrical signal. The amplifier 122 is connected to the impedance measuring circuit 121, and the amplifier 122 is used to amplify the biological impedance signal. The processor 123 is connected to the amplifier 122, and the processor 123 is used to obtain the biological ECG according to the biological impedance signal amplified by the amplifier 122.

The communication module 13 and the on-board communication module 33 may include a wired communication module and a wireless communication module, which are not limited herein.

After receiving the ECG signal from the on-board communication module 33, the on-board processor 32 can control the on-board screen 31 to display the ECG signal.

The on-board processor 32 can also transmit the ECG signal to the on-board operating system 341 in the on-board memory 34. The on-board operating system 341 can call up the ECG detection application 343 in the on-board application database 342 to analyze whether the ECG signal shows abnormalities. If the ECG signal is abnormal, the user can be accordingly reminded by the ECG detection application 343.

The on-board processor 32 can also transmit the ECG signal to the mobile terminal 40 through the on-board communication module 33. An ECG detection application (not shown in the figure) can be stored in the mobile terminal 40 to analyze whether the ECG signal is abnormal. If the ECG signal is abnormal, the mobile terminal 40 can remind the user.

The ECG detection module 10 and the steering wheel 20 of the present disclosure can obtain the ECG signal of the user through the bioelectrical signal of the user, and in time remind the user when the ECG signal of the user shows abnormalities, so as to improve the safety of driving.

Those of ordinary skill in the art should realize that the above embodiments are only used to illustrate the present disclosure, but not to limit the present disclosure. As long as they are within the essential spirit of the present disclosure, the above embodiments are appropriately made and changes fall within the scope of protection of the present disclosure.

What is claimed is:

1. An electrocardiogram (ECG) detection module appliable for a steering wheel, and comprising an ECG detection device, the ECG detection device comprising:
a conductive adhesive film arranged on a surface of the steering wheel and configured to obtain a bioelectric signal of a hand of a user; and
a support member arranged between the steering wheel and the conductive adhesive film, configured to cause the conductive adhesive film to adhere to the steering wheel, and configured to increase a contact area between the conductive adhesive film and the hand of the user;
wherein the support member is elastic.

2. The ECG detection module of claim 1, wherein the support member comprises a substrate, the substrate is arranged between the conductive adhesive film and the steering wheel, and the substrate is configured to cause the conductive adhesive film to adhere to the steering wheel.

3. The ECG detection module of claim 2, wherein the support member further comprises an elastic material, the elastic material is arranged between the substrate and the steering wheel, and configured to increase the contact area between the conductive adhesive film and the hand of the user.

4. The ECG detection module of claim 3, wherein the conductive adhesive film is imprinted on the support member by heat transfer.

5. The ECG detection module of claim 1, wherein the ECG detection device is stretched by thermoforming and imprinted on the steering wheel.

6. The ECG detection module of claim 1, wherein the ECG detection device further comprises an impedance measuring circuit, the impedance measuring circuit is connected to the conductive adhesive film, and the impedance measuring circuit is configured to obtain a biological impedance signal according to the bioelectric signal.

7. The ECG detection module of claim 6, wherein the ECG detection device further comprises an amplifier, the amplifier is connected to the impedance measurement circuit, and the amplifier is configured to amplify the biological impedance signal.

8. The ECG detection module of claim 7, wherein the ECG detection device further comprises a processor, and the processor is connected to the amplifier and configured to obtain a biological ECG according to the biological impedance signal.

9. A steering wheel comprising:
a main body; and
an electrocardiogram (ECG) detection module comprising an ECG detection device, the ECG detection device comprising:
a conductive adhesive film arranged on a surface of the steering wheel, and configured to obtain a bioelectric signal of a hand of a user; and
a support member arranged between the steering wheel and the conductive adhesive film, configured to cause the conductive adhesive film to adhere to the steering wheel, and configured to increase a contact area between the conductive adhesive film and the hand of the user;
wherein the support member is elastic.

10. The steering wheel of claim 9, wherein the ECG detection device is symmetrically arranged on a partial area of the steering wheel, and a remaining area of the steering wheel is provided with a coating material.

11. The steering wheel of claim 9, wherein the support member comprises a substrate, the substrate is arranged between the conductive adhesive film and the steering wheel, and the substrate is configured to cause the conductive adhesive film to adhere to the steering wheel.

12. The steering wheel of claim 11, wherein the support member further comprises an elastic material, the elastic material is arranged between the substrate and the steering wheel, and the elastic material is configured to increase the contact area between the conductive adhesive film and the hand of the user.

13. The steering wheel of claim 12, wherein the conductive adhesive film is imprinted on the support member by heat transfer.

14. The steering wheel of claim 9, wherein the ECG detection device is stretched by thermoforming and imprinted on the steering wheel.

15. The steering wheel of claim 9, wherein the ECG detection device further comprises an impedance measuring circuit, the impedance measuring circuit is connected to the conductive adhesive film, and the impedance measuring circuit is configured to obtain a biological impedance signal according to the bioelectric signal.

16. The steering wheel of claim 15, wherein the ECG detection device further comprises an amplifier, the amplifier is connected to the impedance measurement circuit, and the amplifier is configured to amplify the biological impedance signal.

17. The steering wheel of claim 16, wherein the ECG detection device further comprises a processor, and the processor is connected to the amplifier and configured to obtain a biological ECG according to the biological impedance signal.
